(19) United States
(12) Patent Application Publication
(10) Pub. No.: US 2009/0157068 A1
(43) Pub. Date: Jun. 18, 2009

(54) INTRAOPERATIVE ELECTRICAL CONDUCTION MAPPING SYSTEM

(76) Inventors: Faouzi KALLEL, San Jose, CA (US); Alfred R. CANTU, Pleasanton, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/241,465

(22) Filed: Sep. 30, 2008

Related U.S. Application Data
(60) Provisional application No. 60/976,770, filed on Oct. 1, 2007.

(51) Int. Cl. A61B 18/18

(52) U.S. Cl. 606/33; 600/547

(57) ABSTRACT

A medical probe comprises an elongated body, a first membrane extending laterally from a longitudinal portion of the elongated body, a tissue ablative element carried by the elongated body, and at least one diagnostic electrode disposed on the first membrane. The tissue ablative element is configured for delivering energy along the longitudinal portion of the elongated body to create a linear lesion. The medical probe may further comprise a second membrane extending laterally from the longitudinal portion of the elongated body opposite the first second membrane, and at least another diagnostic electrode disposed on the second membrane.

This application claims priority to Provisional Application Ser. No. 60/976,770 entitled “Intraoperative Electrical Conduction Mapping System” filed Oct. 1, 2007, which is incorporated herein by reference.

### FIELD OF THE INVENTION

The present inventions generally relate to systems and methods for providing therapy to a patient, and more particularly to systems and methods for ablating heart tissue of the patient.

### BACKGROUND OF THE INVENTION

In electrophysiological therapy, tissue ablation is used to treat cardiac rhythm disturbances in order to restore the normal function of the heart. Normal sinus rhythm of the heart begins with the sinoatrial node (or “SA node”) generating a depolarization wave front that propagates uniformly across the myocardial tissue of the right and left atria to the atrioventricular node (or “AV node”). This propagation causes the atria to contract in an organized manner to transport blood from the atria to the ventricles. The AV node regulates the propagation delay to the atrioventricular bundle (or “HIS” bundle), after which the depolarization wave front propagates uniformly across the myocardial tissue of the right and left ventricles, causing the ventricles to contract in an organized manner to transport blood out of the heart. This conduction system results in the described, organized sequence of myocardial contraction leading to a normal heartbeat.

Sometimes, aberrant conductive pathways develop in heart tissue, which disrupt the normal path of depolarization events. For example, anatomical obstacles in the atria or ventricles can disrupt the normal propagation of electrical impulses. These anatomical obstacles can cause the depolarization wave front to degenerate into several circular wavelets that circulate about the obstacles. These wavelets, called “reentry circuits,” disrupt the normal activation of the atria or ventricles. As a further example, localized regions of ischemic myocardial tissue may propagate depolarization events slower than normal myocardial tissue. The ischemic region, also called a “slow conduction zone,” creates errant, circular propagation patterns, called “circus motion.” The circus motion also disrupts the normal depolarization patterns, thereby disrupting the normal contraction of heart tissue. The aberrant conductive pathways create abnormal, irregular, and sometimes life-threatening heart rhythms, called arrhythmias. An arrhythmia can take place in the atria, for example, as in atrial tachycardia (AT), atrial fibrillation (AFIB), or atrial flutter (AF). The arrhythmia can also take place in the ventricle, for example, as in ventricular tachycardia (VT).

In treating these arrhythmias, it is essential that the location of the sources of the aberrant pathways (called substrates) be located. Once located, the tissue in the substrates can be destroyed, or ablated, by heat, chemicals, or other means of creating a lesion in the tissue, or otherwise can be electrically isolated from the normal heart circuit. On the basis of electrophysiology mapping of the atria and identification of macro-reentrant circuits, a surgical approach for treating atrial arrhythmia was developed, which effectively creates an electrical maze in the atrium and precludes the ability of the atria to fibrillate. Briefly, in this procedure commonly referred to as the MAZE III procedure, strategic incisions are performed to prevent atrial reentry circuits from forming and allow sinus impulses to activate the entire atrial myocardium, thereby preserving atrial transport function postoperatively. While the MAZE III procedure has proven effective in treating atrial arrhythmia, this operational procedure requires open-heart surgery and the introduction of substantial incisions within the interior chambers of the heart. Consequently, various other techniques have been developed to interrupt atrial fibrillation and restore normal sinus rhythm.

One such technique is the strategic ablation of heart tissue through ablation catheters, which involves intravenously introducing catheters within the chambers of the heart and endocardially creating transmural ablation lesions within the myocardial tissue. For example, as part of the treatment for certain categories of atrial fibrillation, it may be desirable to create a curvilinear lesion around or within the ostia of the pulmonary veins (PVs), and a linear lesion connecting one or more of the PVs to the mitral valve. In one treatment for AF, a linear lesion may be created along the cavotricuspid isthmus (CTI) between the inferior vena cava (IVC) and the tricuspid annulus (TA). When creating lesions within the myocardial tissue for the purpose of preventing conduction from one side of the lesion to the other side of the lesion, it is important to ensure that the lesion is continuous so that there are no “break-through” points within the lesion and a complete conduction block has been achieved.

In one known technique, illustrated in FIGS. 1 and 2, and described in Maruyama, et al. “Mapping-Guided Ablation of the Cavotricuspid Isthmus: A Novel Simplified Approach to Radiofrequency Catheter Ablation of Isthmus-Dependent Atrial Flutter, Heart Rhythm, Vol. 3, No. 6, June 2006, an ablation catheter 2 is used to create a linear ablation lesion L along the CTI, and a separate mapping catheter 4 and pacing catheter 6 are used to confirm that a complete bidirectional conduction block was formed across the CTI by the linear lesion L. In particular, as shown in FIG. 1, the mapping catheter 4, which in the illustrated embodiments carries eight linearly displaced electrodes, was placed parallel to and on one side of the linear lesion L, and the pacing catheter 6 is placed in the coronary sinus (CS), so that the linear lesion L is interposed between the mapping and pacing catheters 4, 6. The site is then paced with the pacing catheter 6 to emulate a clockwise atrial fibrillation reentry circuit, while recording signals on the eight electrodes of mapping catheter 4 to determine the existence and location of any break-through points (shown by arrows). If any exist, the CTI is then ablated again at the locations of the break-though points. As shown in FIG. 2, the mapping catheter 4 is then placed parallel to and on the other side of the linear lesion L, and the pacing catheter 6 is placed in the low lateral right atrium (LLRA), so that the linear lesion is interposed between the mapping and pacing catheters 4, 6. The site is then paced with the pacing catheter 6 to emulate a counterclockwise atrial fibrillation reentry circuit, while recording signals on the eight electrodes of the mapping catheter 4 to determine the existence and locations of any break-through points (shown by arrows). If any exist, the CTI is then ablated again at the locations of the break-though points.

Most approved ablation catheters now utilize radio frequency (RF) energy as the ablating energy source. Accordingly, a variety of RF based catheters and power supplies are currently available. Other tissue ablation instruments, which utilize other energy sources, have been developed. Microwave frequency energy, for example, has long been recognized as an effective energy source for heating biological tissues and has seen use in such hyperthermia applications as cancer treatment and preheating of blood prior to infusions. Currently, microwave surgical probes are introduced through the chest of the patient, either percutaneously or laparoscopically, or through a surgical opening, and then operated to create transmural lesions from the epicardial surface.

Whether transmural ablation lesions are formed endocardially or epicardially, separate mapping probes are used to confirm bidirectional conduction blocks of linear lesions. This requires the tedious placement of mapping probes parallel and adjacent to the linear lesions, and maintaining the mapping probes at these positions and orientations throughout the procedure, thereby increasing procedure time.

Accordingly, there remains for an ablation/mapping system that can more efficiently create linear ablation lesions and confirm bi-directional conduction blocks of such lesion.

### SUMMARY OF THE INVENTION

In accordance with a first aspect of the present inventions, a medical probe assembly is provided. The medical probe assembly comprises an ablation sheath including an elongated sheath body, an ablation lumen longitudinally extending within the sheath body, a first membrane extending laterally from a longitudinal portion of the sheath body, and at least one diagnostic electrode disposed on the membrane. The ablation sheath may be configured to be intravascularly introduced into a patient (e.g., in contact with endocardial tissue) or extravascularly introduced into a patient (e.g., in contact with the epicardial tissue). In an optional embodiment, the ablation sheath further includes a second membrane extending laterally from the longitudinal portion of the sheath body opposite the first second membrane, and at least another diagnostic electrode disposed on the second membrane. If a plurality of diagnostic electrodes is provided on the first and/or second membranes, they may be longitudinally aligned substantially parallel to the sheath body.

The medical probe assembly further comprises an ablation device slidably disposed within the ablation lumen. The ablation device includes an elongated member and a tissue ablative element disposed on the elongated member. The tissue ablative element is configured for delivering energy along the longitudinal portion of the sheath body to create a linear lesion. In one embodiment, the energy delivered by the tissue ablative element is electromagnetic energy. For example, the tissue ablative element may comprise a microwave antenna.

In one embodiment, the medical probe assembly further comprises an actuating mechanism configured for allowing a user to longitudinally translate the ablation device within the ablation lumen. In another embodiment, the sheath body has a substantially flat side through which the energy from the tissue ablative element is delivered, and the first membrane and/or second membrane laterally extend from the sheath body in a plane substantially parallel to the flat side. In another embodiment, the first membrane is configured for longitudinally sliding along the sheath body. In still another embodiment, the ablation sheath may further comprise one or more vacuum ports disposed on the first membrane.

The medical probe assembly can be incorporated into a medical system that comprises a source of ablation energy coupled to the tissue ablative element, and a tissue mapping processor coupled to the at least one diagnostic electrode. The medical probe assembly can also be used in a method that comprises delivering the energy from the tissue ablative element along the longitudinal portion of the sheath body to create the linear lesion within tissue (e.g., myocardial tissue), recording electrical signals within the tissue along the linear lesion via the at least one diagnostic electrode, and analyzing the recorded electrical signals to determine the existence of any conduction break-through points along the linear lesion. The method may further comprise redelivering the energy from the tissue ablation element to complete the linear lesion adjacent any determined conduction break-through points.

In accordance with a second aspect of the present inventions, a medical probe is provided. The medical probe comprises an elongated body, a first membrane extending laterally from a longitudinal portion of the elongated body, a tissue ablative element carried by the elongated body, and at least one diagnostic electrode disposed on the first membrane. The detailed features of the medical probe and its use in systems and methods can be the same as those described above, with the exception that the ablative element need not be slidably disposed within the elongated body. For example, the ablative element can be configured to selectively create discrete lesions in the tissue to form a continuous lesion, or the ablative element can otherwise be long enough to create a continuous lesion having the desired length within the tissue.

Other features of the present invention will become apparent from consideration of the following description taken in conjunction with the accompanying drawings.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIGS. 1 and 2 are plan views of a prior art method of endocardially creating a continuous linear lesion along a cavotricuspid isthmus of the heart and verifying bi-directional conduction blocking by the lesion;

FIG. 3 is a plan view of one embodiment of an electrophysiology system constructed in accordance with the present inventions;

FIG. 4 is a partially cutaway perspective view of an ablation probe used in the system of FIG. 3;

FIG. 5 is a cross-sectional view of a probe assembly used in the system of FIG. 3, taken along the line 5-5;

FIG. 6 is a partially cutaway plan view illustrating the a lower surface of the probe assembly of FIG. 5;

FIG. 7a-7d are plan views illustrating a method of creating a continuous linear lesion with the probe assembly of FIG. 5;

FIG. 8 is a partially cutaway perspective view of an alternative probe assembly that can be used in the system of FIG. 3; and

FIGS. 9-13 are perspective views of one method of using the system of FIG. 3 to epicardially create a continuous linear lesion along the tissue of a heart and verify bi-directional conduction blocking by the lesion.

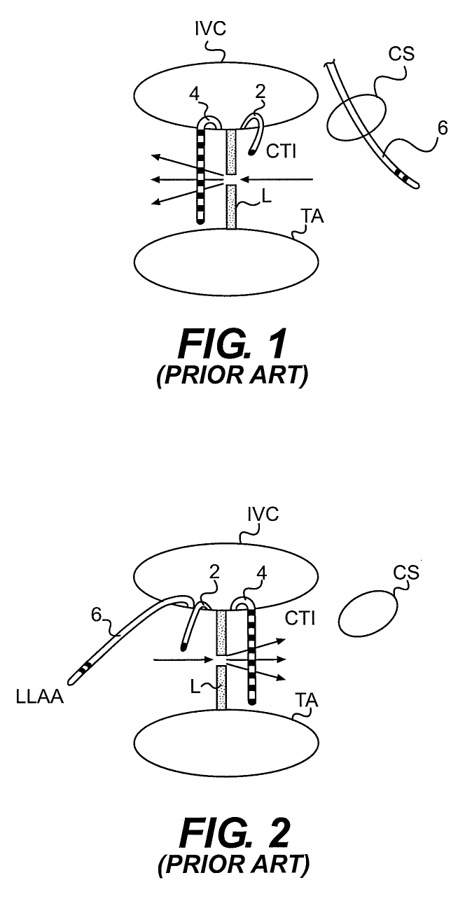

### DETAILED DESCRIPTION OF THE PREFERRED EMBODIMENTS

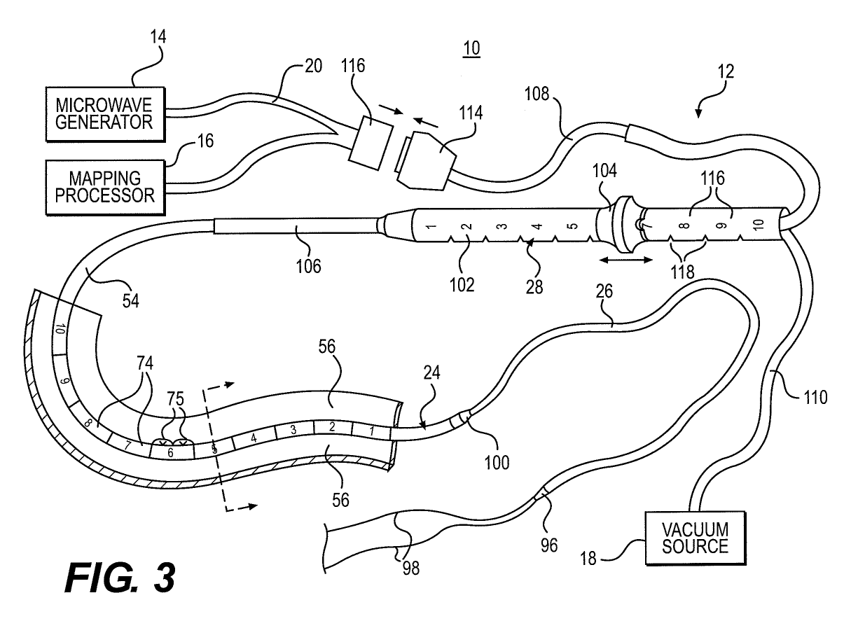

Referring to FIG. 3, an exemplary electrophysiology system 10 constructed in accordance with the present inventions is shown. The system 10 may be used in any bodily region of a patient, but in the illustrated embodiment, is described as being used to ablate tissue of the heart, and in the illustrated embodiment, to epicardially ablate myocardial tissue. The system 10 generally comprises an electrophysiology probe assembly 12 configured for forming linear lesions within tissue and for detecting electrical signals from tissue, a source of ablation energy 14 for delivering ablation energy to the probe assembly 12, a mapping processor 16 for processing the electrical signals detected by the probe assembly 12, a source of vacuum 18 for stabilizing the probe assembly 12 relative to the tissue, and a cable assembly 20 for coupling the source of ablation energy 14 and mapping processor 16 to the probe assembly 12. Although the source of ablation energy 14, mapping processor 16, and vacuum source 18 are shown as discrete components, they can alternatively be incorporated into a single integrated device.

The ablation probe assembly 12 is configured for being percutaneously introduced through the chest of a patient or introduced through a surgical opening within the chest of a patient into contact with the epicardial tissue of the heart, and operated to convey electromagnetic ablation energy into the epicardial tissue to ablate targeted regions within the myocardial tissue, e.g., to treat atrial fibrillation of the heart. For example, as will be described in further detail below, a linear ablation may be formed along the isthmus between the pulmonary veins and the mitral annulus. While the ablation probe assembly 12 is described herein as being non-vascularly introduced into a patient, it should be appreciated that the scope of the invention may include probe assemblies that are designed to be introduced vascularly into a patient, e.g., to be placed into contact with the endocardial tissue. Further details regarding the ablation probe assembly 12 will be discussed further below.

The source of electromagnetic ablation energy 14, and in particular, a conventional microwave generator, is configured for providing the ablation energy to the probe assembly 12. The microwave generator 14 may include all of the standard components that microwave generators typically have, including a housing that contains all of the ablation circuitry and various control mechanisms, indicators that allow a user to control the parameters of the tissue ablation, including frequency, amplitude, and duration, and a connector port that mates with the probe assembly 12 via the cable assembly 20. The ablative energy may alternatively be derived from a laser source, a cryogenic source, an ultrasonic source, or a radiofrequency source, to name a few.

When using microwave energy for tissue ablation, the optimal frequencies are generally in the neighborhood of the optimal frequency for heating water. By way of example, frequencies in the range of approximately 800 MHz to 6 GHz work well. Currently, the frequencies that are approved by the Federal Communication Commission (FCC) for experimental clinical work includes 915 MHz and 2.45 GHz. Therefore, a microwave generator having the capacity to generate microwave energy at frequencies in the neighborhood of 2.45 GHz may be chosen. A conventional magnetron of the type commonly used in microwave ovens is utilized as the generator. It should be appreciated, however, that any other suitable microwave power source could be substituted in place, and that the explained concepts may be applied at other frequencies like about 434 MHz or 5.8 GHz (ISM band).

The mapping processor 16 is configured to detect, process, and record electrical signals within the heart via the probe assembly 12. Based on the electrical signals detected by the probe assembly 12, the mapping processor 16 outputs electrocardiograms (ECGs) to a display (not shown), which can be analyzed by the user to can determine the existence and location of any break-through points in the linear lesion formed by the probe assembly 12. In an optional embodiment, the display may graphically show any gap that exists across the linear lesion. The vacuum source 18 may take the form of any standard suction source available in operating rooms, such as a wall-mounted or stand-alone suction source. Suction may be provided by the vacuum source 18 at a negative pressure of between 200-600 mm Hg, with 400 mm Hg being preferred.

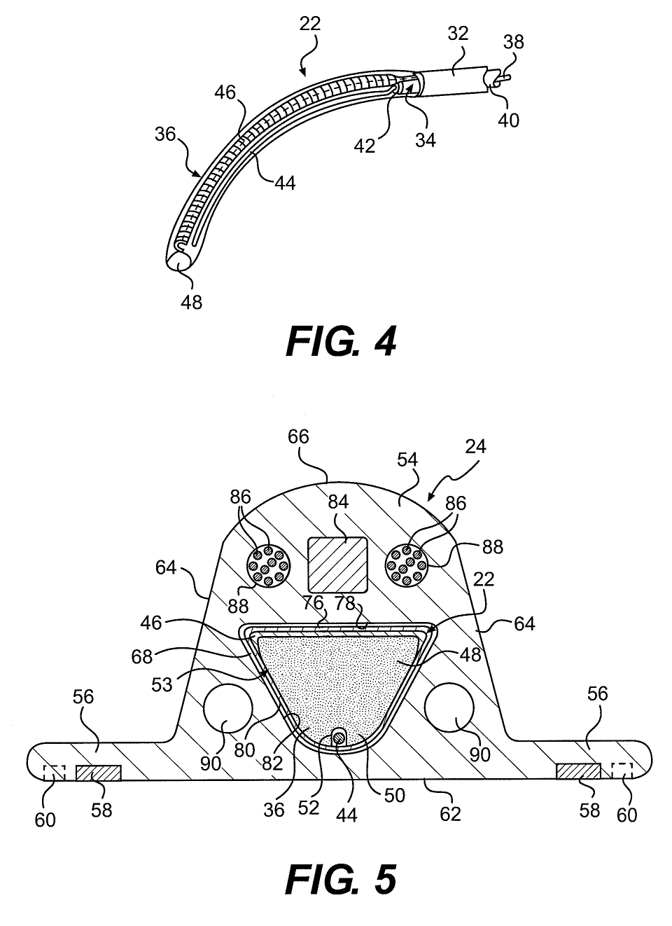

The probe assembly 12 generally comprises an elongated ablation probe 22 (shown in FIG. 4), an elongated ablation sheath 24 in which the ablation probe 22 is disposed, a guide member 26 extending from the distal end of the ablation sheath 24, a handle assembly 28 extending from the proximal end of the ablation sheath 24.

Referring specifically to FIGS. 4 and 5, the ablation probe 22 includes a flexible outer tubing 32, a transmission line 34 extending through the outer tubing 32, and an ablative element 36 coupled to the distal end of the transmission line 34. The outer tubing 32 may be composed of any suitable material, such as medical grade polyolefins, fluoropolymers, or polyvinylidene fluoride. By way of example, Pebax® resins from Autochem of Germany have been used with success for the outer tubing 32. In the illustrated embodiment, the transmission line 34 is a coaxial transmission line that includes an inner conductor 38, an outer conductor 40, and a dielectric material 42 disposed between the inner and outer conductors 38, 40.

In the illustrated embodiment, the ablative element 36 is a microwave ablative element. Alternatively, the ablative element 36 is a cryogenic, ultrasonic, laser, or radio frequency (RF) ablative element, to name a few. The ablative element 36, as a microwave ablative element, is configured to directionally emit a majority of the electromagnetic field from one side thereof. In particular, the ablative element 36 includes a directed component that cooperates with the energy source to control the direction and emission of the ablation energy. This helps preserve the non-targeted tissue surrounding the targeted tissue regions. Correspondingly, the resultant electromagnetic field includes components of the originally generated field and the redirected electromagnetic field. Further, the use of a directional electromagnetic field has several potential advantages over conventional energy delivery structures that generate uniform fields about the longitudinal axis of the ablative element 36.

For example, in the microwave application, by forming a more concentrated and directional electromagnetic field, deeper penetration of biological tissues is enabled, and the targeted tissue region may be ablated without heating as much of the surrounding tissues and/or blood. Additionally, since substantial portions of the radiated ablative energy is not emitted in the air or absorbed in the blood or the surrounding tissues, less power is generally required from the power source, and less power is generally lost in the transmission line. To this end, the ablative element 36 includes a flexible antenna 44 for generating the electromagnetic field, a flexible reflector 46 as a directive component for redirecting a portion of the electromagnetic field to one side of the antenna 44 opposite the reflector 46, and a flexible insulator 48 encapsulating the antenna 44 and reflector 46. As a result, the ablative element 36 directionally transmits ablation energy through a window 50.

The insulator 48 is composed of a good, low-loss dielectric material that is relatively unaffected by microwave exposure, and thus is capable of transmission of the electromagnetic field therethrough. Moreover, the material of the insulator 48 has a low water absorption, so that it is not itself heated by the microwaves. Furthermore, the material of the insulator 48 should be capable of substantial flexibility without fracturing or breaking. Such materials include moldable fluoropolymer like Teflon®, silicone, polyethylene, polyimide, etc. The insulator 48 comprises a groove 52 adapted to receive the antenna 44 therein. The groove 52 acts to restrict the lateral movement of the antenna 44 during use, maintaining the antenna 44 fixed with respect to the reflector 46. As the ablation probe 22 deflects during use, the groove 52 allows the antenna 44 to translate therein. Thus, during such deflection, the groove 52 serves to restrict lateral movement of the antenna 44 and maintain proper orientation of the reflector 46 relative to the antenna 44.

The antenna 44 is a simple exposed wire type antenna composed of a suitable metallic material. By way of example, copper or silver-plated metal work well. Further, the diameter of the antenna 44 may vary to some extent based on the particular application of the probe assembly 12 and the type of material chosen. For example, wire diameters between about 0.010 to about 0.020 inches work well. In the illustrated embodiment, the diameter of the antenna 44 is about 0.013 inches. It should be appreciated that, while the antenna 44 is described and illustrated as a longitudinally extending wire, a wide variety of other antenna geometries may be used as well. By way of example, helical coils, flat printed circuit antennas, and other antenna geometries will work as well. As will be appreciated by those familiar with antenna design, the electromagnetic field generated by the antenna 44 will be generally consistent with the length of the antenna 44. That is, the length of the electromagnetic field is generally constrained to the longitudinal length of the antenna 44. Therefore, the length of the electromagnetic field may be selected by selecting the length of the antenna 44.

The antenna 44 is coupled directly or indirectly to the inner conductor 38 of the coaxial transmission line 34. A direct connection between the antenna 44 and the inner conductor 38 may be made in any suitable manner, such as soldering, brazing, ultrasonic welding, or adhesive bonding. In other embodiments, the antenna 44 can be formed from the inner conductor 38 of the transmission line 34, itself. In some implementations, it may be desirable to indirectly couple the antenna 44 to the inner conductor 38 through a passive component, such as a capacitor, an inductor, or a stub tuner, for example, to provide better impedance matching between the antenna assembly and the transmission line 34, thereby maximizing the passage of electromagnetic ablative energy to the antenna 44.

In a preferred embodiment, the antenna 44 is placed proximate to the outer peripheral surface of the insulator 48 in order to achieve effective energy transmission between the antenna 44 and the targeted tissue via the ablation window 50. This is best achieved by placing the antenna 44 proximate to the ablation window 50. In particular, the longitudinal axis of the antenna 44 is offset from, but parallel to, the longitudinal axis of the inner conductor 38 of the transmission line 34 in a direction away from the reflector 46. The offset nature of the antenna 44 with respect to the longitudinal axis of the inner conductor 38 allows for the deposition of a greater amount of electromagnetic ablative energy within the target tissue. By way of example, placing the antenna 44 between about 0.010 to about 0.020 inches away from the outer peripheral surface of the insulator 48 works well. In the illustrated embodiment, the antenna 44 is about 0.013 inches away from the outer peripheral surface of the insulator 48. However, it should be noted that this is not a requirement, and that the position of the antenna 44 may vary according to the specific design of the probe assembly 12.

The reflector 46 has two main functions: to prevent the passage of ablative energy therethrough and to reflect at least a portion of the ablative energy in a desired predetermined direction. To this end, the reflector 46 is positioned adjacent and generally parallel to the antenna 44. The edges of the reflector 46 curve over the top surface of the insulator 48 and toward the window 50, resulting in a curvilinear cross-sectional geometry more suitable for directing at least a portion of the ablative electromagnetic energy generated by the antenna 44 towards the window 50. Alternatively, the reflector 46 has an arcuate or meniscus (crescent) shape. The reflector 46 is preferably configured to permit at most a 180° circumferential ablation pattern from the antenna 44. In fact, it has been discovered that arc angles greater than about 180° are considerably less efficient. More preferably, the arc angle of the radiation pattern is in the range of about 90° to about 120°. While the reflector 46 is shown and described as having a curvilinear cross-sectional shape, it will be appreciated that a plurality of forms may be provided to accommodate different antenna shapes or to conform to other external factors necessary to complete a surgical procedure. For example, any flared shape that opens toward the antenna 44 may work well, regardless of whether it is curvilinear or rectilinear.

In the illustrated embodiment, the reflector 46 is composed of a material that restricts the passage of electromagnetic energy therethrough, while allowing for the desired flexibility. To this end, the reflector 46 is composed of a highly conductive material, such as copper, silver or gold. To increase the flexibility of the ablative element 36, the reflector 46 is relatively flexible. One particularly suitable material for such a reflector is a braided conductive mesh, although a thin metallic foil that is pleated or folded can be used as well. In an alternative embodiment, the reflector 46 is composed of a dielectric material having a dielectric constant different than that of the material from which the insulator 48 is composed. Indeed, a strong reflection of electromagnetic energy is observed when the electromagnetic wave reaches an interface created by the two materials with a different dielectric constant. For example, a ceramic loaded polymer can have a dielectric constant between 15 and 55, while the dielectric constant of a fluropolymer, like Teflon®, can be between 2 and 3. Such an interface would create a strong reflection of the electromagnetic energy and act as a semi-reflector.

The reflector 46 may be attached to the top surface of the insulator 48 using any suitable means, such as epoxy bonding. Alternatively, the reflector 46 may be press fit or otherwise formed atop the insulator 48. A thin insulating sheath 53, preferably shrink tubing or the like, is applied over the entire surface of the insulator 48, holding the reflector 46 and the insulator 48 in place with respect to each other. As should be readily understood, the insulating sheath 53 also acts to retain the antenna 44 within the groove 52, and allows for closer placement of the antenna 44 to the active side of the ablation sheath 24. While the flexible reflector 46 is shown attached atop the top flat surface of the insulator 48, the reflector 46 may be embedded within the insulator 48.

The proximal end of the reflector 46 is preferably coupled to the outer conductor 40 of the transmission line 34, which serves to better define the electromagnetic field generated by the ablative element 36. That is, the radiated field is better confined along the antenna 44 to one side when the reflector 46 is electrically connected to the outer conductor 40 of the transmission line 34. The connection between the reflector 46 and the outer conductor 40 may be made in any suitable manner, such as soldering, brazing, ultrasonic welding, or adhesive bonding. In other embodiments, the reflector 46 can be formed from the outer conductor 40 of the transmission line 34 itself. To reduce undesirable electromagnetic coupling between the antenna 44 and the reflector 46, the reflector 46 is preferably offset from the antenna 44 by a certain distance. It has been found that the minimum distance between the reflector 46 and the antenna 44 may be between about 0.020 to about 0.030 inches in order to reduce the coupling. However, the distance may vary according to the specific design of the probe assembly 12.

Further details of the ablation probe 22, as well as alternative ablation probes that can be used in the probe assembly 12, are disclosed in U.S. Pat. Nos. 6,471,696 and 7,033,352, and U.S. patent application Ser. No. 10/348,256, which is expressly incorporated herein by reference.

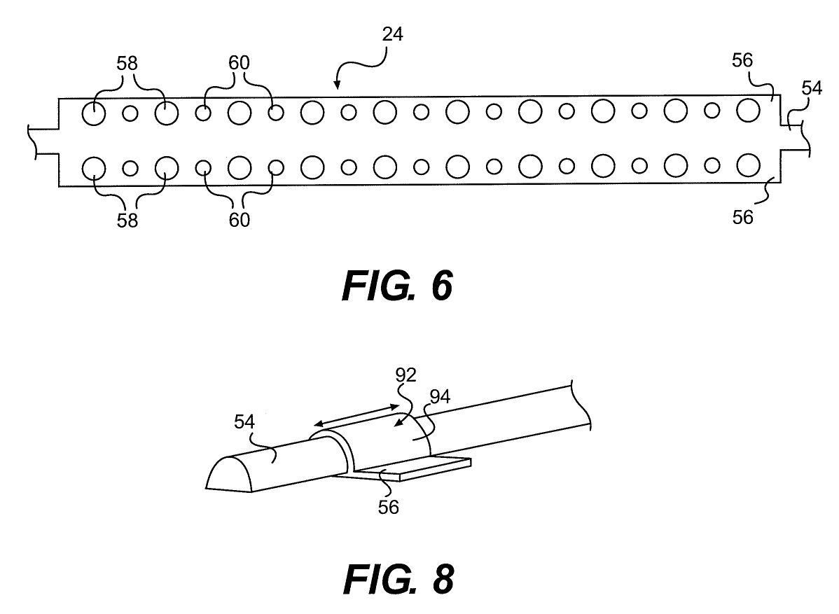

The ablation sheath 24 comprises a flexible elongated body 54, a pair of membranes 56 laterally extending from the ablation energy emitting region of the sheath body 54 in opposite directions, a plurality of diagnostic electrodes 58 (best shown in FIG. 6) disposed on the membranes 56, and a plurality of vacuum ports 60 (best shown in FIG. 6) disposed on the membranes 56. In the illustrated embodiment, the energy emitting region of the sheath body 54 is the region in which ablation element 36 translates. The ablation sheath 24 serves to define an ablation path along which the ablation probe 22 is guided. To this end, the sheath body 54 is capable of conforming to the curvature of a body organ; e.g., the curved epicardial tissue surface of the heart. The sheath body 54 is also substantially transparent to electromagnetic energy in the microwave frequency range. Moreover, the sheath body 54 is substantially unaffected by the ablation energy emitted by the ablative element 36 of the ablation probe 22.

Thus, the material of the sheath body 54 should exhibit selected properties, such as a low loss tangent, low water absorption or low scattering coefficient to name a few, to be unaffected by the ablation energy. To this end, the sheath body 54 may be composed of an expanded polytetrafluoroethylene (ePTFE), which provides the requisite flexibility to pass through various body cavities or take on curvatures associated with various body organs, and which provides superior radial strength and is resistant to lateral compression, such that the translation of the ablation probe 22 within the ablation sheath 24 is not compromised. Such a material also has the advantage of improved torque, such that when a user rotates the proximal portion of the ablation sheath 24 external to the patient's body, the remaining portion of the ablation sheath 24 within the body is encouraged to rotate into the same orientation.

While the sheath body 54 may have any suitable cross-sectional geometric shape, the cross-sectional geometric shape is adapted in the illustrated embodiment to encourage proper orientation of the sheath 24 relative to the target tissue, and to provide enhanced thermal protection with respect to the non-target tissue adjacent or in the general vicinity of the target tissue. To this end, and as best shown in FIG. 5, the sheath body 54 has a substantially flat bottom side 62 (or active ablation side), angled lateral sides 64, and a curved top side 66. Notably, due to its flat geometry, uniform contact between the active ablation side 62 and the target tissue tends to be maintained. The curvature of the top side 66 of the sheath body 54 allows for dynamic contact with surrounding tissues, while minimizing undesirable damage based on such contact.

The ablation sheath 24 further comprises an ablation lumen 68 longitudinally extending through the sheath body 54 for slidably receiving the ablation probe 22. Accordingly, in one embodiment, the ablation sheath 24 functions to guide or position the ablative element 36 of the ablation probe 22 properly along an ablation path defined by the sheath 24 as the ablative element 36 is advanced through the ablation lumen 68. By positioning the ablative element 36, which is preferably adapted to emit a directional ablation field, at one of a plurality of positions incrementally along the ablation path in the ablation lumen 68, a single continuous or plurality of spaced-apart lesions can be formed.

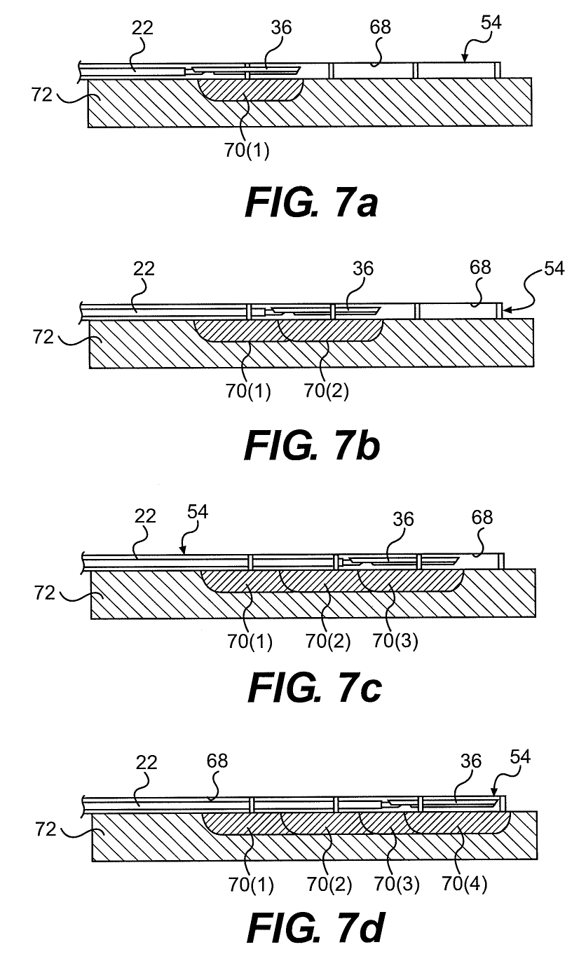

For example, as illustrated in FIGS. 7a-7d, as the ablative element 36 of the ablation probe 22 is incrementally advanced through the ablation lumen 68, overlapping lesion sections 70(1)-70(4) are formed within tissue 72 by the directional ablation field. Collectively, a continuous lesion or series of lesions can be formed along the active ablation side 62 of the sheath body 54, which is positioned adjacent to or in contact with the targeted tissue 72. These transmural lesions may thus be formed in any shape on the target tissue region 72, such as rectilinear, curvilinear, or circular in shape. Further, depending upon the desired ablation line patterns, both opened and closed path formation can be constructed. In other instances, the length of the ablative element 36 may be sufficient to extend along the entire ablation path, so that only a single ablation sequence is necessary.

The ablation sheath 24 further comprises a plurality of reference markings 74 located on the top side 62 of the sheath body 54. In the illustrated embodiment, ten reference markings 74 are used, although in alternative embodiments, less reference markings 74 (e.g., four) or more reference markings 74 can be utilized. Each reference marking 74 defines a position along the sheath body 54 within which the ablative element 36 of the ablation probe 22 travels. As will be described in further detail below, these markings 74 are correlated to position indicators located on the handle assembly 24, so that the user knows the exact position of the ablative element 36 translating with the sheath body 54 at any given time during use. Each reference marking 74 may define an incremental energy emitting segment 75 that is substantially equal to the length of the ablative element 36, but preferably is slightly shorter than the length of the ablative element 36 to allow for overlapping of individual lesions during use, resulting in the creation of a continuous lesion. For example, the length of the incremental energy emitting segment 75 defined by each reference marking 74 can be in the range of 1 cm-4 cm. In the illustrated embodiment, the length of each energy emitting segment 75 is 2 cm.

While the reference markings 74 are shown as comprising numerical values as identifiers, any suitable means of identifying or distinguishing the positions may be used. For example, roman numerals or other alphanumeric or graphical symbols may be used. The reference markings 74 can be disposed on the sheath body 54 using any suitable means. For example, the reference markings 74 may be formed through an inking process, providing a contrast between the reference markings 74 and the sheath body 54. Alternatively, the reference markings 74 may also comprise various materials with fluoresce, allowing the user to view the reference markings 74 on a monitor (not shown) as part of a fluoroscopy system. If the reference markings 74 do fluoresce, they may be further be adapted to indication rotational orientation of the sheath body 54 as well.

Notably, when using directional ablation fields, such as those emitted by the ablative element 36 of the ablation probe 22, it is important to provide a mechanism for continuously aligning the directional field of the ablative element 36 with the targeted tissue region for ablation. Thus, the directional field should be continuously aligned with the predetermined active side 62 of the sheath body 54, which is in contact with the targeted tissue region, as the ablative element 36 is advanced through the ablation lumen 68. In this manner, a physician may determine that, once the predetermined active side 62 of the sheath body 54 is properly oriented and positioned adjacent or in contact against the targeted tissue, the directional component of the ablative element 36 will then be automatically aligned with the targeted tissue as it is advanced through the ablation lumen 68.

To this end, the transverse cross-sectional geometries of the ablative element 36 and ablation lumen 68 are configured in manner that continuously position the directive ablation energy towards the active side 62 of the sheath body 54. In the illustrated embodiment, the cross-sectional geometries of the ablative element 36 and ablation lumen 68 are generally D-shaped and substantially similar in dimension. In particular, the ablative element 36 and ablation lumen 68 are shaped to have substantially flat top surfaces 76, 78 and substantially curved bottom surfaces 80, 82, thereby encouraging/maintaining proper orientation of the ablative element 36 relative to the ablation lumen 68. It will be appreciated, however, that any geometric configuration may be used to ensure continuous and aligned insertion of the ablative element 36 within the ablation lumen 68 can be used. As another example, the ablative element 36 and interior wall of the ablation lumen 68 may include a key member and corresponding receiving groove (not shown) or the like.

To facilitate shape retention, the ablation sheath 24 further comprises a shape retaining member 84 extending longitudinally through the distal portion of the sheath body 54 where shape retention is necessary. The retaining member 84 generally extends substantially parallel and adjacent to the ablation lumen 68 to reshape the predetermined active side 62 once the restraining forces are removed from the ablation sheath 24. Alternatively, the retaining member 84 may be malleable, thereby allowing the user to conform the ablation sheath 24 to a specific orientation.

Alternatively, the retaining member 84 may be adapted to retain the specific length of the sheath body 54, such that a user can be assured of the position of the ablation probe 22 translating within the ablation lumen 68. For example, if the sheath body 54 is composed of porous ePTFE, a material which offers tremendous resistance to radial compression, but can longitudinally expand and contract, the retaining member 84 would act to limit the longitudinal expansion and contraction of the sheath body 54 over its length. In illustrated embodiment, the retaining member 84 is composed of a shape-memory material, such as nitinol, and has a diameter in the range of 0.020-0.050 inches. For cases where a more flexible sheath body 54 is desired, the retaining member 84 may be composed of a more flexible material, such as silicone, or plastics or metals which have the desired degree of flexibility, for example. It should be noted that the retaining member 84 may be adapted to having a differing resistance along its length.

The ablation sheath 24 further comprises a plurality of signal wires 86 longitudinally extending within the sheath body 54. The signal wires 86 are composed of a suitably electrically conductive material that exhibits the desired properties of low resistance, corrosion resistance, flexibility, and strength. The signal wires 86 are electrically insulated from each other to prevent degradation of the detected signals, and are respectively connected to the diagnostic electrodes 58 disposed on the membranes 56, thereby carrying the electrical signals detected by the electrodes 58 to the proximal end of the sheath body 54. The connection between the electrodes 58 and the signal wires 86 may be made in any suitable manner such as soldering, brazing, ultrasonic welding or adhesive bonding. In the illustrated embodiment, the signals wires 86 are shown as extending through two lumens 88 located along the respective sides of the sheath body 54, although they can also extend through a single lumen or more than two lumens. Alternatively, the signals wires 86 may extend within the retaining member 84, for example, if the retaining member 84 is composed of a material, such as silicone. The ablation sheath 24 further comprises two suction lumens 90 extending through the sheath body 54. As will be described in further detail below, the suction lumens 90 are in fluid communication with the suction ports 60 located on the membranes 56.

In the illustrated embodiment, the membranes 56 laterally extend from the sheath body 54 in a plane that is parallel to the flat active side 62 of the sheath body 54, and the diagnostic electrodes 58 are disposed on the underside of the membranes 56, such that the diagnostic electrodes 58 can be placed into contact with the epicardial tissue without moving the active side 62 through which the ablation energy is conveyed. Preferably, the plane in which the membranes 56 extend is the same plane in which the flat active side 62 of the sheath body 54 is disposed, such that a continuous surface is formed along the active side 62 and underside of the membranes 56. Alternatively, the plane in which the membranes 56 extend can be offset from the plane in which the flat active side 62 of the sheath body 54 is disposed (e.g., the plane of the membrane 50 can extend through the center of the sheath body 54).

In the illustrated embodiment, the membranes 56 are composed of a continuous layer of material, although alternatively, the membranes 56 may be composed of a porous material, mesh, or braid. The membranes 56 are configured to be placed into a compact, low-profile geometry by, e.g., rolling or folding the membranes 56 around the sheath body 54, and maintained in this low-profile geometry by applying a radial compressive force to the rolled or folded membranes 56, such as the force that would be applied by the lumen of a delivery device. To this end, the material from which the membranes 56 are composed is relatively thin (e.g., 0.1 mm to 2 mm, although 1 mm or less is most preferred) and has a relatively low-stiffness. Exemplary materials are low-stiffness silicone, ePTFE, or urethane. Due to these properties, the membranes 56 can be more easily collapsed into a low-profile geometry.

In an optional embodiment, the membranes 56 spring open from their collapsed low-profile geometry upon the release of a radial compressive force, e.g., when the sheath body 54 exits the delivery device. In this case, a spring element (not shown) composed of a relatively stiff and resilient material, such as stainless steel, a metallic and polymer material, or a high-stiffness urethane or silicone, can be disposed on or in the membranes 56 to urge them into their planar shape. In alternative embodiments, the spring element can be composed of a shape memory material, such as nitinol, so that the membranes 56 assume the planar geometry in the presence of a defined temperature, such as, e.g., body temperature.

The electrodes 58 essentially measure whether there is any electrical activity (or electrophysiological signals) to one or the other side of the sheath body 54. In the illustrated embodiment, the diagnostic electrodes 58 are arranged on the respective membranes 56 as single columns that are substantially parallel to the longitudinal axis of the sheath body 54. In this manner, the arrangement of electrodes on linear mapping catheters are emulated (of course, with the need to actually properly orienting the mapping catheters adjacent the sheath body 54), so that break-though points within the linear ablation lesion formed by the ablative element 36 of the ablation probe 22 can be detected, and bi-directional blocking of the conduction through the ablation lesion can be confirmed, as will be described in further detail below.

Preferably, the electrodes 58 in each column are equally spaced a relatively small distance from each other to maximize the sensing resolution. For example, the spacing between the electrodes 58 can be in the range of 0.5 mm and 5 mm. The width (or diameter) and the length of each electrode 58 may vary to some extent based on the particular application of the probe assembly 12 and the type of material chosen for the electrode 58. Furthermore, in the preferred embodiment where microwave is used as the ablative energy, the electrodes 58 are preferably dimensioned to minimize electromagnetic field interference, for example, the capturing of the microwave field produced by the antenna 44. While the illustrated embodiment only shows two columns of 10 electrodes (i.e., a pair of electrodes 58 for each energy emitting segment 75 defined a reference marking 74) for purposes of brevity, because it may be desirable to space the electrodes 58 as closely as possible, each column may have more than 10 electrodes. For example, if each energy emitting segment 75 is 2 cm, and an electrode spacing of 5 mm within each column is desired, each column may have 40 electrodes (4 electrodes for each reference marking 74).

In alternative embodiments, multiple columns of electrodes or some other electrode pattern can be located on each membrane 50. In addition, when a strong electrical activation signal is detected or inter-electrode impedance is measured when two or more of the electrodes 58 are applied, contact between the active side 62 of the sheath body 54 can be assessed. Once the physician has properly situated and oriented the ablation sheath 24, he or she may commence advancement of the ablative element 36 through the ablation lumen 68. Additionally, the electrodes 58 may be applied to map the heart tissue prior to an ablation procedure, as well as be used to monitor the patient's condition during the ablation process.

The electrodes 58 can be disposed on the membranes 56 using known deposition processes, such as sputtering, vapor deposition, ion beam deposition, electroplating over a deposited seed layer, or a combination of these processes. Alternatively, the electrodes 58 can be disposed onto the membranes 56 as a thin sheet or foil of electrically conductive metal. Or, the electrodes 58 can be discrete elements that are embedded into the membranes 56, such that they lie flush with the surface of the membranes 56. The electrodes 58 can be composed of a suitable electrically conductive and biocompatible material, e.g., platinum, platinum/iridium, stainless steel, gold, or combinations of alloys of these materials. In the illustrated embodiment, the electrodes 58 are circular, but can formed as other geometric shapes, such as rectangular or elliptical. In other embodiments, the electrodes 58 can be formed from the signal wires 86 themselves. Forming the electrodes 58 from the signal wires 86, or out of wire in general, is particularly advantageous because the size of wire is generally small and therefore the electrodes 58 may be positioned closer together.

The suction ports 60 essentially stabilize and ensure that the membranes 56, and thus the electrodes 58, are in firm contact with the epicardial tissue. That is, when a vacuum is applied to the suction ports 60 via the suction lumens 90, the surfaces of the membranes 56 will naturally secure themselves to the adjacent tissue on which the suction operates. In the illustrated embodiment, the suction ports 60, like the electrodes 58, are arranged on the respective membranes 56 as single columns that are substantially parallel to the longitudinal axis of the sheath body 54. Although the suction ports 60 are shown as being interposed between electrodes 58, the suction ports 60 can be arranged in any pattern that facilitates the firm contact between the electrodes 58 and epicardial tissue.

In the embodiment illustrated in FIG. 6, the membranes 56 are affixed to the sheath body 54 and extend the entire length of the sheath body 54 in which the ablative element 36 of the ablation probe 22 slides, and in this case, from the first reference marking 74 all the way to the tenth reference marking 74. In an optional embodiment illustrated in FIG. 8, a slidable membrane assembly 92 is provided. In this embodiment, the slidable membrane assembly 92 comprises a collar 94 slidably disposed about the sheath body 54, with the membranes 56 (only one shown) laterally extending from the sheath body 54 via the collar 94. Thus, it can be appreciated that the membrane assembly 92 can be slid along the sheath body 54 anywhere between, and including, the first and tenth reference markings 74. In this manner, the length of the membranes 56, as well as the number of electrodes 58 and associated vacuum ports 60, can be substantially reduced, since the membrane assembly 92, without moving the sheath body 54, can be moved to the reference marking or markings 74 where the linear ablation is currently be formed. The length of the membranes 56 can, for example, be equal to the length of the two incremental longitudinal segments 75, e.g., 4 cm, which is the typical length of a linear ablation.

Further details of the ablation sheath 24, as well as alternative ablation sheaths that can be used in the probe assembly 12, are disclosed in U.S. patent application Ser. No. 10/348,256, which is expressly incorporated herein by reference.

The guide member 26 is adapted to better guide the ablation sheath 24 in the proper orientation adjacent or proximate to the target tissue to be ablated. The guide member 26 can be formed in any suitable fashion to facilitate proper placement of the ablation sheath 24. The guide member 26 is constructed from any suitable material, allowing the necessary flexibility to be guided through tight areas within a human body, e.g., around the pulmonary veins between the pericardium and the epicardial tissue of the heart. While the guide member 26 may be composed of any suitable material, e.g., silicone, ePTFE, Pebax®, or polyurethane, the guide member 26 is preferably composed of a porous ePTFE. The guide member 26 can be any suitable length for a given ablation procedure. For example, the guide member 26 may have a suitable length to encircle a body organ, for example, the heart, such that the ablation sheath 24 is not specifically manipulated during initial placement of the guide member 26. In this way, undesirable damage to the guide member 26 is minimized.

As shown, the guide member 26 terminates in a blunt distal end 96 as a means to protect surrounding tissues during the guiding process. The blunt distal end 96 may be composed of any suitable material, e.g., stainless steel or thermoplastic elastomer. The guide member 26 may further comprise suture lines 98 connected to the distal end 96 to facilitate users in guiding the guide member 26. For example, a user can blindly advance the distal end 96 of the guide member 26 around a bodily object until the suture lines 98 once again appear. A medical tool may then be used to grasp the suture lines 98, allowing for the complete encircling of the object the guide member 26, and ultimately, the guide sheath 24. The guide member 26 is radially sized to allow for guiding through narrow openings naturally occurring or created by the user during use. If the guide member 26 is radially sized differently from the sheath body 54, an enlarger/reducer may be utilized to facilitate the transition from the sheath body 54 to the guide member 26. For example, if the guide member 26 is radially sized smaller than the sheath body 54, as generally shown in FIG. 3, a reducer 100 may be utilized to facilitate the transition from the larger diameter sheath body 54 to the smaller diameter guide member 26. The reducer 100 may be composed of any suitable material, e.g., stainless steel or thermoplastic elastomer.

Further details of the guide member 26 are disclosed in U.S. patent application Ser. No. 10/348,256, which is expressly incorporated herein by reference.

Referring specifically to FIG. 3, the handle assembly 28 comprises a handle body 102, an actuator ring 104 slidably disposed on the handle body 102, a rigid tube 106 distally extending from the handle body 102, an electrical cable 108 extending from the proximal end of the handle body 102, and a suction conduit 110 extending from the proximal end of the handle body 102.

The handle body 102 is preferably composed of a durable and rigid material, such as medical grade plastic, and is ergonomically molded to allow a physician to more easily manipulate the probe assembly 12. The actuator ring 104 slides relative to the handle body 102 (shown by double arrow) to translate the ablative element 36 of the ablation probe 22 within the ablation lumen 68 of the ablation sheath 24, so that the ablative element 36 can be selectively located at one of the desired positions. The rigid tube 104 is suitably mounted within a bore (not shown) at the distal end of the handle body 102 using suitable means, such as epoxy bonding, and the proximal end of the sheath body 54 is suitably mounted within the distal end of the tube 106.

The electrical cable 108 is electrically connected within the handle body 102 to the transmission line 34 extending through the probe body 32 and the signal wires 86 extending through the sheath body 54. The electrical cable 108 includes an electrical connector 112 that mates with a corresponding electrical connector 114 of the cable assembly 20, which in turn, is coupled to the microwave generator 14 and mapping processor 16, thereby placing the microwave generator 14 into electrical communication with the ablative element 36 of the ablation probe 22, and the mapping processor 16 into electrical communication with the diagnostic electrodes 58 located on the membranes 56 of the ablation sheath 24. The suction conduit 110 is fluidly connected within the handle body 102 to the suction lumens 90 extending through the sheath body 54. The suction conduit 110 includes an vacuum connector (not shown) that mates directly with the vacuum source 18, thereby placing the vacuum source 18 into fluid communication with the suction ports 60 located on the membranes 56 of the ablation sheath 24.

The handle assembly 28 further comprises a plurality of position indicators 116 located on the handle body 102 for indicating the position of the ablative element 36 within the ablation sheath 24; that is, position indicator 1 indicates that the ablative element 36 is at the corresponding reference marker 1 on the ablation sheath 24, position indicator 2 indicates that the ablative element 36 is at the reference marker 2 on the ablation sheath 24, and so on. The handle assembly 28 further comprises a plurality of indentations 118 suitably formed along the handle body 102 and adapted to provide tactile feedback to the user when the ablative element 36 is properly placed. These indentations 118 are adapted to cooperate with an associated mechanism on the actuator ring 104, such as a ball and spring mechanism (not shown). As the actuator ring 104 is translated about the handle body 102, the force of the associated mechanism engages one of the indentations 118. The position indicators 116 and associated indentations 118 are spaced apart along the handle body 102 a distance substantially equal to the length of the ablative element 36. More preferably, the spacing is defined as having a length slightly shorter than the length of the ablative element 36 to allow for overlapping of individual lesions during use, resulting in the creation of a continuous long lesion. Further details discussing the handle assembly 28 are disclosed in U.S. patent application Ser. No. 10/348,256, which is expressly incorporated herein by reference.

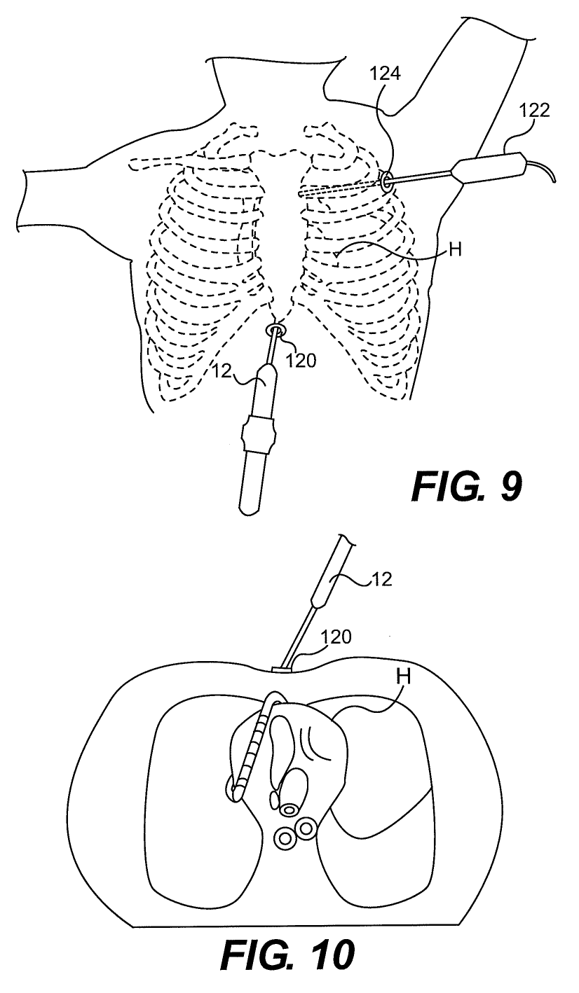

Having described the structure and arrangement of the electrophysiology system 10, its use in minimally invasively ablating the epicardium of a beating heart via an endoscopic procedure in order to treat an arrhythmia of the heart will now be described. Referring first to FIGS. 9 and 10, at least one access port 120 is formed in the thorax of the patient. In the illustrated method, the access port 120 is a subxyphoid port that provides access to the posterior epicardial tissue of the heart H. A dissection tool (not shown) or the like may be utilized to facilitate access the pericardial cavity. For instance, the pericardium may be dissected to enable access to the epicardium of the heart. The pericardial reflections may be dissected in order to allow the positioning of the probe assembly 12 around the pulmonary veins if desired. Another dissection tool (not shown) may also be utilized to puncture the pericardial reflection located in proximity to the pulmonary vein. After the pericardial reflection is punctured, the probe assembly 12 can be positioned around the heart, as best shown in FIG. 10, in order to produce the ablation pattern used to treat the heart arrhythmia. For example, the probe assembly 12 may be inserted though the access port 120 via the aid of an introducer (not shown), while visualizing the insertion process with an endoscopic device 122 positioned through another access port 124. Notably, when the probe assembly 12 is introduced through the access port 120, the membranes 56 may be wrapped around the sheath body 54 or otherwise folded into a low-profile geometry to ease introduction of the probe assembly 12 through the access port 124.

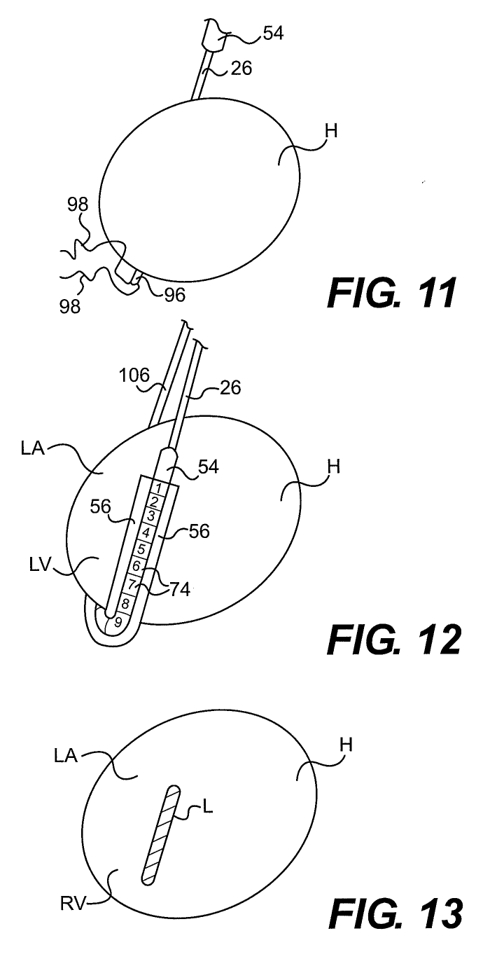

When introducing the probe assembly 12, the blunt distal end 96 of the guide member 26 can initially be advanced to a side of the heart H opposite to the access port 120, as illustrated in FIG. 11. At this point, an additional medical device (not shown), perhaps introduced through a third access port (not shown), may be utilized to grasp the sutures 98 and retracted, further guiding otherwise positioning, the energy emitting region of the sheath body 54 against the targeted tissue of the heart, as illustrated in FIG. 12. In this case, the targeted tissue is the isthmus between the pulmonary veins of the left atrium LA and the mitral annulus between the left atrium LA and left ventricle LV. As shown, the reference markers 74 are shown, meaning that the active side 62 is properly located in contact with the epicardial tissue.

Once properly positioned, the microwave generator 14 is operated to selectively deliver the ablative microwave energy from the ablative element 36 of the ablation probe 22 through the active side 62 of the sheath body 54, while the ablative element 36 is periodically translated within the ablation lumen 68 of the ablation sheath 24, thereby creating a long continuous lesion L within the myocardial tissue, as shown in FIG. 13. Notably, the length of the lesion L will correspond to the position indicators 116 selected by the user on the handle assembly 28 (e.g., in this case positions 4-7). Of course, additional continuous lesions L can be created at different regions of the heart H, depending on the specific electrical activity patterns to be blocked.

Notably, during the ablation process, the vacuum source 18 is operated to create suction at the suction ports 60, thereby creating firm contact between the epicardial tissue and the membranes 56. As a result, the diagnostic electrodes 58, as well as the active side 62 of the sheath body 54, are placed into firm contact with the epicardial tissue. Without moving the active side 62, the mapping processor 16 can be operated to measure electrical activity via the diagnostic electrodes 58 during or after the ablation process to confirm that a bidirectional conduction block is created across the lesion. For example, electrical activity readings can be taken along each side of the lesion by measuring bipolar electrical activity of adjacent electrodes 58 along the electrode columns and analyzing the double potentials of each reading, as disclosed in Maruyama, et al. “Mapping-Guided Ablation of the Cavotricuspid Isthmus: A Novel Simplified Approach to Radiofrequency Catheter Ablation of Isthmus-Dependent Atrial Flutter, Heart Rhythm, Vol. 3, No. 6, June 2006. If any break-through points along the lesion L are found, they can be ablated without moving the ablation sheath 24 simply by positioning the ablative element 36 at the reference markers 74 corresponding to the locations where the break-through points were found and redelivering the microwave ablative energy from the ablative element 36 at those locations. Pacing can either be accomplished by generating a pulse on one of the diagnostic electrodes 58 in the column opposite the electrode column currently be used to record the bipolar electrical activity, or if pacing from one of the diagnostic electrodes 58 would generate undesirable artifacts as a result of its close proximity to the recording ones of the electrodes 58, a separate pacing probe can be introduced into contact with the epicardial tissue to generate the pacing pulses.

Although particular embodiments of the present invention have been shown and described, it will be understood that it is not intended to limit the present invention to the preferred embodiments, and it will be obvious to those skilled in the art that various changes and modifications may be made without departing from the spirit and scope of the present invention. Thus, the present inventions are intended to cover alternatives, modifications, and equivalents, which may be included within the spirit and scope of the present invention as defined by the claims.

What is claimed is:

1. A medical probe assembly, comprising:
an ablation sheath including an elongated sheath body, an ablation lumen longitudinally extending within the sheath body, a first membrane extending laterally from a longitudinal portion of the sheath body, and at least one diagnostic electrode disposed on the first membrane; and
an ablation device slidably disposed within the ablation lumen, the ablation device including an elongated member and a tissue ablative element disposed on the elongated member, the tissue ablative element configured for delivering energy along the longitudinal portion of the sheath body to create a linear lesion.

2. The medical probe assembly of claim 1, wherein the ablation sheath further includes a second membrane extending laterally from the longitudinal portion of the sheath body opposite the first second membrane, and at least another diagnostic electrode disposed on the second membrane.

3. The medical probe assembly of claim 1, wherein the at least one diagnostic electrode comprises a plurality of electrodes longitudinally aligned substantially parallel to the sheath body.

4. The medical probe assembly of claim 1, wherein the energy delivered by the tissue ablative element is electromagnetic energy.

5. The medical probe assembly of claim 4, wherein the tissue ablative element comprises a microwave antenna.

6. The medical probe assembly of claim 1, further comprising an actuating mechanism configured for allowing a user to longitudinally translate the ablation device within the ablation lumen.

7. The medical probe assembly of claim 1, wherein the sheath body has a substantially flat side through which the energy from the tissue ablative element is delivered, and wherein the first membrane laterally extends from the sheath body in a plane substantially parallel to the flat side.

8. The medical probe assembly of claim 1, wherein the first membrane is configured for longitudinally sliding along the sheath body.

9. The medical probe assembly of claim 1, wherein the ablation sheath further comprises at least one vacuum port disposed on the first membrane.

10. A medical system, comprising:
the medical probe assembly of claim 1;
a source of ablation energy coupled to the tissue ablative element; and
a tissue mapping processor coupled to the at least one diagnostic electrode.

11. A method of using the medical probe of claim 1, comprising:
delivering the energy from the tissue ablative element along the longitudinal portion of the sheath body to create the linear lesion within tissue;
recording electrical signals within the tissue along the linear lesion via the at least one diagnostic electrode; and
analyzing the recorded electrical signals to determine the existence of any conduction break-through points along the linear lesion.

12. The method of claim 11, further comprising redelivering the energy from the tissue ablation element to complete the linear lesion adjacent any determined conduction break-through points.

13. The method of claim 11, wherein the tissue is myocardial tissue.

14. A medical probe, comprising:
an elongated body;
a first membrane extending laterally from a longitudinal portion of the elongated body;
a tissue ablative element carried by the elongated body, the tissue ablative element configured for delivering energy along the longitudinal portion of the elongated body to create a linear lesion; and
at least one diagnostic electrode disposed on the first membrane.

15. The medical probe of claim 14, further comprising:
a second membrane extending laterally from the longitudinal portion of the elongated body opposite the first second membrane; and
at least another diagnostic electrode disposed on the second membrane.

16. The medical probe of claim 14, wherein the at least one diagnostic electrode comprises a plurality of electrodes longitudinally aligned substantially parallel to the elongated body.

17. The medical probe of claim 14, wherein the energy delivered by the tissue ablative element is electromagnetic energy.

18. The medical probe of claim 14, wherein the tissue ablative element comprises a microwave antenna.

19. The medical probe of claim 14, wherein the elongated body has a substantially flat side through which the energy from the tissue ablative element is delivered, and wherein the first membrane laterally extends from the elongated body in a plane substantially parallel to the flat side.

20. The medical probe assembly of claim 14, wherein the first membrane is configured for longitudinally sliding along the elongated body.

21. The medical probe assembly of claim 14, wherein further comprises at least one vacuum port disposed on the first membrane.

22. A medical system, comprising:
the medical probe assembly of claim 14;
a source of ablation energy coupled to the tissue ablative element; and
a tissue mapping processor coupled to the at least one diagnostic electrode.

23. A method of using the medical probe of claim 14, comprising:
delivering the energy from the tissue ablative element along the longitudinal portion of the elongated body to create the linear lesion within tissue;
recording electrical signals within the tissue along the linear lesion via the at least one diagnostic electrode; and
analyzing the recorded electrical signals to determine the existence of any conduction break-through points along the linear lesion.

24. The method of claim 23, further comprising redelivering the energy from the tissue ablation element to complete the linear lesion adjacent any determined conduction break-through points.

25. The method of claim 23, wherein the tissue is myocardial tissue.
